(19) United States
(12) Patent Application Publication
(10) Pub. No.: US 2007/0235025 A1
(43) Pub. Date: Oct. 11, 2007

(54) Baby hernia belt

(76) Inventor: Rachelle Fink, Alma, KS (US)

Correspondence Address: JAMES RAY & ASSOCIATES, 2640 PITCAIRN ROAD, MONROEVILLE, PA, 15146, US

(21) Appl. No.: 11/713,140

(22) Filed: Mar. 2, 2007

Related U.S. Application Data
(60) Provisional application No. 60/790,907, filed on Apr. 10, 2006.

(51) Int. Cl. A61F 5/03

(52) U.S. Cl. 128/96.1

(57) ABSTRACT

A belt for protecting an infant with a herniated belly button. The belt comprises a substantially soft sleeve like member for encircling an infant so as to cover such herniated belly button. The substantially soft sleeve like member having a first predetermined shape and formed of a first predetermined material. A stiff-ribbed portion made of a second predetermined material and having a second predetermined shape and a predetermined width is designed for insertion into the substantially soft sleeve like member for providing support to such herniated belly button. Hook and loop material is used for securing one end of the belt to another portion of the belt around a body of such infant so that such belt is sufficiently tight so as to securely hold the belly button in, while preventing sores and discomfort to such infant.

### CROSS REFERENCE TO RELATED APPLICATIONS

This application is closely related to and claims benefit from U.S. Provisional Application Ser. No. 60/790,907 filed Apr. 11, 2006.

### FIELD OF THE INVENTION

The present invention relates, in general, to a hernia belt, and, more particularly, the present invention relates to a hernia belt which will hold the belly in, while preventing sores and discomfort to the baby who has an umbilical hernia.

### BACKGROUND OF THE INVENTION

An umbilical hernia is a bulge around the belly button. This can occur if the muscle around the navel does not close completely. This can cause distress to the parent which is very understandable. Often, these hernias are caused or aggravated by a particular activity or movement. One present solution to treat the pediatric herniated belly button is to wrap an Ace bandage around the area. Although this solution can prevent some patients from undergoing surgery, this restrictive device does not please the baby very well. It is difficult and awkward to apply, as well as difficult to retain the proper pressure on the hernia.

Thus, it would be advantageous if there were a means available for a baby with an umbilical hernia that would alleviate the problem and still not be a discomfort to the baby and also would be easy to apply and retain the proper pressure.

### SUMMARY OF THE INVENTION

In a first aspect the present invention provides a belt for protecting an infant with a herniated belly button. The belt comprises a substantially soft sleeve like member for encircling an infant so as to cover such herniated belly button. The substantially soft sleeve like member having a first predetermined shape and a first predetermined width and formed of a first predetermined material. A stiff-ribbed portion made of a second predetermined material and having a second predetermined shape and a second predetermined width is designed for insertion into the substantially soft sleeve like member for providing support to such herniated belly button. There is a means for securing one end of the belt to another portion of the belt around a body of such infant so that such belt is sufficiently tight so as to securely hold the belly button in, while preventing sores and discomfort to such infant.

### OBJECTS OF THE INVENTION

It is, therefore, one of the primary objects of the present invention is to provide a belt for securely holding a belly button in on an infant with a herniated belly button.

Another object of the present invention is to provide a belt for securely holding a belly button which will not cause sores or discomfort to such infant.

Still another object of the present invention is to provide a belt for securely holding a belly button in that is secured by means of hook and loop material.

Yet another object of the present invention is to provide a belt for securely holding a belly button in that uses dress ribbing to provide support to the belly button.

Another object of the present invention is to provide a belt for securely holding a belly button which is not restrictive to such infant.

These and various other objects and advantages of this invention will become apparent after a full reading of the following detailed description, particularly, when read in conjunction with the attached drawings as described below and the appended claims.

### BRIEF DESCRIPTION OF THE DRAWINGS

FIG. 1 is a perspective view of a soft sleeve like member of the belt according to an embodiment of the invention.

FIG. 2 is a perspective view of the dress ribbing that is used to provide stiffness for the sleeve like member shown in FIG. 1.

FIG. 3 is a perspective view of the belt as rolled up as it would be around an infant.

FIG. 4 is a perspective view of the belt shown in FIG. 3 around the body of an infant.

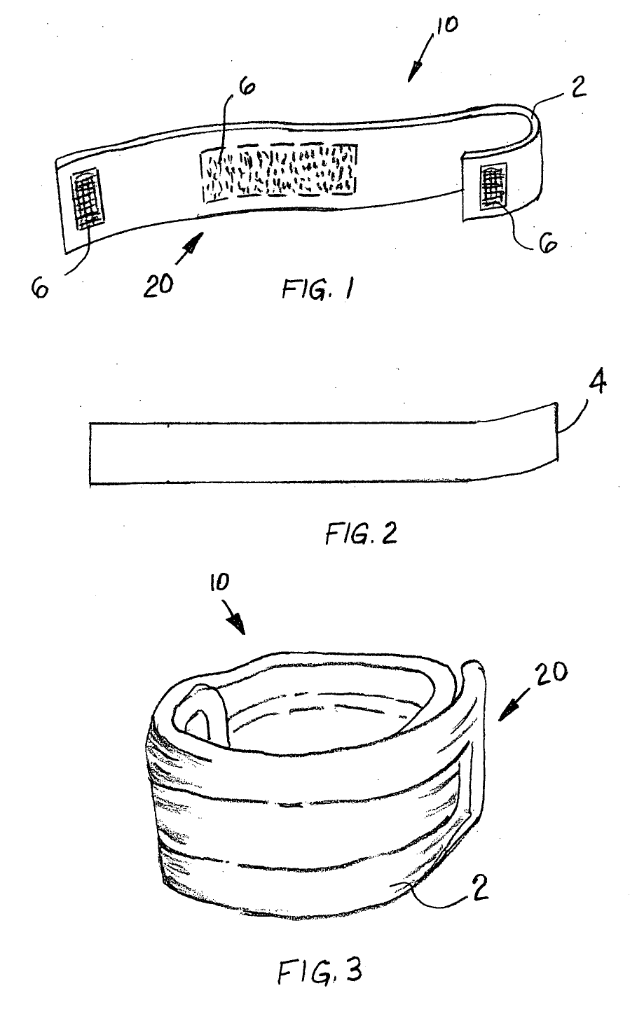

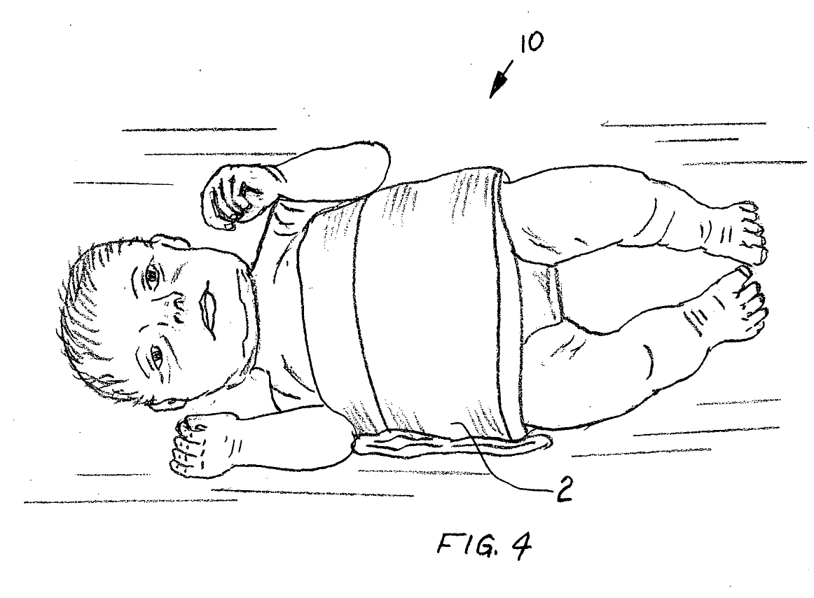

### BRIEF DESCRIPTION OF THE PRESENTLY PREFERRED AND ALTERNATE EMBODIMENTS OF THE INVENTION

Prior to proceeding with the more detailed description of the present invention it should be noted that, for the sake of clarity, identical components which have identical functions have been designated by identical reference numerals throughout the several views illustrated in the drawings.

In a first aspect the present invention provides a belt, generally designated 10, for protecting an infant with a herniated belly button. The belt 10 comprises a substantially soft sleeve like member 2 for encircling an infant so as to cover such herniated belly button. The substantially soft sleeve like member 2 has a first predetermined shape and a first predetermined width and is formed of a first predetermined material. A stiff-ribbed portion 4 is made of a second predetermined material and has a second predetermined shape and a second predetermined width for insertion into the substantially soft sleeve like member 2 for providing support to such herniated belly button. There is a means, generally designated 20, for securing one end of the belt 10 to another portion of the belt 10 around a body of such infant so that such belt 10 is sufficiently tight so as to securely hold the belly button in, while substantially eliminating sores or discomfort to such infant that accompanies many of the products currently being used.

The second predetermined material for the stiff-ribbed portion 4 is a dress ribbing 4. Such dress ribbing 4 can be found at most fabric stores. The predetermined width of such dress ribbing 4 is approximately two inches. The width is sufficient to cover the belly button and provide support to the belly button.

The first predetermined material for the sleeve like member 2 is a soft fleece material. The soft fleece material provides softness next to the skin of the infant and does not irritate the skin and therefore helps to alleviate discomfort or sores that have been associated with previously used bandages.

The securing means 20 that is used to secure the ends of the belt 10 and provide a tight fit includes hook and loop material 6. The hook and loop material 6 has a first strip which has a predetermined length and a predetermined width and is disposed on one side of the belt 10 while a second strip of such hook and loop material 6 that is substantially identical to the first strip is disposed on an opposite side of the belt 10. Such first and such second strips are disposed lengthwise along a longitudinal axis of the belt. This provides a large area for securement so as to accommodate different sized infants. Such first strip and such second strip are used for securing the belt 10 tightly against an infant's belly so as securely hold such belly button in.

The securing means 20 further includes a third strip of hook and loop material 6 disposed closely adjacent one end of the belt and a fourth strip of hook and loop material 6 disposed closely adjacent the other end of the belt on an opposite side from the third strip. The third and fourth strips are disposed lengthwise on an axis that is perpendicular to the longitudinal axis of the belt. Such third strip and such fourth strip of hook and loop material 6 are for securing the ends of the belt 10 to the first and second strips so that belt is sufficiently tight and the ends are not loose when the belt is placed around an infants belly.

The belt 10 is between about 2 feet and 3 inches and about 2 feet and 9 inches in length so a to accommodate various sized infants. The width of the belt 10 belt is between about 3 and about 4 inches. The belt is wide enough that the stiff-ribbed portion 4 fits inside easily.

The open end portions of the substantially soft sleeve like member 2 after insertion of the stiff ribbed portion 4 are secured by one of snaps, sewing or hook and loop material. Presently it is preferred that such closure is done with snaps or hook and loop material so as to make it easy to remove the stiff-ribbed portion 4 from the sleeve like member 2 for washing the substantially soft sleeve like member 2.

The soft fleece used for the sleeve like member 2 is designed to provide softness next to such infant's skin so as to substantially reduce any discomfort to such infant. Such substantially soft sleeve like member 2 is provided in various colors to make the belt more attractive.

When such belt is wrapped around such infant the herniated belly button is compressed back into an infant's belly and the infant is not restricted by the belt as is the case with some of the presently used products.

While a presently preferred embodiment and alternate embodiments of the present invention has been described in detail above, it should be understood that various other adaptations and/or modifications of the invention can be made by those persons who are particularly skilled in the art without departing from either the spirit of the invention or the scope of the appended claims.

I claim:

1. A belt for protecting an infant with a herniated belly button, said belt comprising:
(a) a substantially soft sleeve like member for encircling an infant so as to cover such herniated belly button, said substantially soft sleeve like member having a first predetermined shape and a first predetermined width and formed of a first predetermined material; and
(b) a stiff-ribbed portion made of a second predetermined material and having a second predetermined shape and a second predetermined width for insertion into said substantially soft sleeve like member for providing support to such herniated belly button;
(c) a means for securing one end of said belt to another portion of said belt around a body of such infant so that said belt is sufficiently tight so as to securely hold a belly button in, while preventing sores and discomfort to such infant.

2. The belt, according to claim 1, wherein said second predetermined material is dress ribbing.

3. The belt, according to claim 1, wherein said second predetermined width is approximately two inches.

4. The belt, according to claim 1, wherein said first predetermined material is a soft fleece material.

5. The belt, according to claim 1, wherein said securing means includes hook and loop material.

6. The belt, according to claim 5, wherein a first strip having a predetermined length and a predetermined width of said hook and loop material is disposed on one side of said belt while a second strip of said hook and loop material substantially identical to said first strip is disposed on an opposite side of said belt, said first and said second strip are disposed lengthwise along a longitudinal axis of said belt.

7. The belt, according to claim 6, wherein said belt further includes a third strip of hook and loop material disposed closely adjacent one end of said belt and a fourth strip of hook and loop material disposed closely adjacent another end of said belt on an opposite side from said third strip, said third and said fourth strips are disposed widthwise on said belt along an axis perpendicular to said longitudinal axis.

8. The belt, according to claim 8, wherein said third strip and said fourth strip engage said first and said second strips for securing ends of said belt so that said belt is tight against the infants belly and said ends are not loose when said belt is placed around an infants belly.

9. The belt, according to claim 8, wherein said first, said second, said third and said fourth strips are used for securing said belt tightly against an infant's belly so as to securely hold such belly button in.

10. The belt, according to claim 1, wherein said belt is between about 2 feet and 3 inches and about 2 feet and 9 inches in length so a to accommodate various sized infants.

11. The belt, according to claim 1, wherein said belt is between about 3 and about 4 inches in width.

12. The belt, according to claim 1, wherein open ends of said substantially soft sleeve like member after insertion of said stiff ribbed portion are secured by one of snaps, sewing, and hook and loop material.

13. The belt, according to claim 12, wherein said ends are secured by hook and loop material.

14. The belt, according to claim 13, wherein said stiff ribbed portion is removable from said substantially soft sleeve like member.

15. The belt, according to claim 1, wherein said substantially soft sleeve like member is provided in various colors.
